(19) United States
(12) Patent Application Publication
(10) Pub. No.: US 2024/0207349 A1
(43) Pub. Date: Jun. 27, 2024

(54) GEL FOR THE REGENERATION OF MUCOSAE

(71) Applicant: POLITECNICO DI MILANO, Milano (IT)

(72) Inventors: Lorenzo SARDELLI, Milano (IT); Caterina MANDELLI, Milano (IT); Francesco BRIATICO VANGOSA, Milano (IT); Paola PETRINI, Milano (IT)

(73) Assignee: POLITECNICO DI MILANO, Milano (IT)

(21) Appl. No.: 18/556,992

(22) PCT Filed: Apr. 26, 2022
(86) PCT No.: PCT/IB2022/053847

§ 371 Date: Oct. 24, 2023

(30) Foreign Application Priority Data
Apr. 26, 2021 (IT) 102021000010433

(51) Int. Cl. A61K 36/886; A61K 9/06; A61K 31/198; A61K 31/375; A61K 31/59; A61K 31/714; A61K 31/732; A61K 33/10; A61K 33/30; A61K 33/38; A61K 35/747

(52) U.S. Cl. CPC A61K 36/886; A61K 9/06; A61K 31/198; A61K 31/375; A61K 31/59; A61K 31/714; A61K 31/732; A61K 33/10; A61K 33/30; A61K 33/38; A61K 35/747

(57) ABSTRACT

The present invention relates to a composition in the form of a gel comprising Aloe vera juice and pectin in a concentration between 0.1 and 3%, preferably of about 0.3%. Optionally, said composition comprises further active ingredients. The present invention also relates to said composition for use in the local treatment of inflammatory states of mucosae.

### BACKGROUND ART

Mucosae are anatomical barriers with the dual function of protecting tissues from the external environment and housing specific types of bacterial flora. Regardless of the anatomical site, the inflammatory states of mucosae are common and difficult to treat, both due to their exposure to pathogens in the external environment and due to the imbalances they produce in the flora. An example is proctitis, i.e., an inflammation of the rectal mucosa, the etiology of which can be Crohn's disease, an ulcerative colitis, or sexually transmitted diseases, including gonorrhea, syphilis, infection with Chlamydia trachomatis, infection with Herpes simplex virus or Cytomegalovirus. Bacteria which are not sexually transmitted, such as Salmonella, or the use of antibiotics depleting the intestinal bacterial flora cause proctitis as well. Another cause is radiation therapy of the rectum or nearby tissues, used to treat prostate or rectal cancer.

To date, antibiotics are the best treatment for proctitis caused by a specific bacterial infection.

To date, there are no effective local treatments for treating inflammatory states of the mucosa, whatever the etiology, except the application of corticosteroids or sucralfate by means of enema.

The anti-inflammatory and antioxidant properties of Aloe vera are widely recognized. Aloe vera also has therapeutic power for mucosal ulcers due to the immunological properties thereof.

Aloe vera contains two distinct sources of juice: i) latex, which is an exudate contained in the pericyclic cells of the vascular bundles located at the junction between the rind (cuticle) and the inner part of the Aloe leaves; ii) mucilage, which is a transparent cellular gel, extracted from the pulp or inner part of the Aloe leaves once the rind (cuticle) has been removed and the exudate (latex) has been moved away.

US2002/0119941 describes the extraction of pectin from Aloe and the ability of this pectin, dissolved in water, to gel in situ, for example after subcutaneous injection, or after topical application.

CN101164583 describes a composition for topical use comprising Aloe latex.

Singh et al., in Annals of Biology, 2014; 30: 705-710 describe a composition comprising Aloe vera and pectin with a high degree of esterification, without controlling the dosages thereof, and operating a heat treatment which inevitably involves an undesired degradation of the high molecular weight pectin.

IT201700035757 describes a composition comprising Aloe vera for use in rectal applications. However, it does not provide any indication about the rheological properties of the composition.

The need for effective local treatments for treating inflammatory states of the mucosa remains strongly felt.

### DESCRIPTION OF THE INVENTION

The present invention relates to a composition in the form of a gel comprising Aloe vera juice, where said juice is mucilage deprived of the latex, pectin, and optionally one or more active ingredients selected from the group comprising: vitamins, amino acids, short-chain fatty acids and/or salts thereof, bacteria, including probiotics, derivatives thereof and products thereof. A process for preparing said composition is also described.

The use of said composition for treating inflammatory states of the mucosa is further described. In a further embodiment, said gel is used as a carrier for the release of trophic factors capable of locally regenerating the mucosa, by way of example, said gel is an enema for the release of trophic factors for the rectal mucosa.

### DESCRIPTION OF THE DRAWINGS

FIG. 1: Graphs representative of the viscosity of the product as a function of the shear rate. (A, comparative) fresh Aloe (circles), pectin (circles, squares, barred triangles) at the indicated concentrations; (B, according to the invention) fresh Aloe (circles), composition comprising the mixture of fresh Aloe and pectin (circles, squares, black triangles) at the indicated concentrations.

FIG. 2: Graphs representative of the viscosity of the product as a function of the shear rate. (A, comparative) commercial Aloe (circles), pectin (circles, squares, barred triangles) at the indicated concentrations; (B, according to the invention) commercial Aloe (circles), composition comprising commercial Aloe and pectin (circles, squares, black triangles) at the indicated concentrations.

FIG. 3: Comparative graph showing the viscosity of a product comprising pectin (white columns) compared to a product comprising the same concentration of pectin and Aloe, commercial (bar columns) or fresh (black columns).

FIG. 4: Correlation between the trend of viscosity and concentration of a product comprising pectin in the absence (white circles) or in combination with Aloe (black circles). (A) fresh Aloe; (B) commercial Aloe.

FIG. 5: Modules G′ and G″ measured at the indicated frequencies in compositions comprising pectin (squares), Aloe (triangles), Aloe+pectin (circles) (A) in the absence of crosslinker and reaction initiator; (B) in the presence of crosslinker; (C) in the presence of crosslinker and reaction initiator.

FIG. 6: Modules G′ and G″ measured at 1 Hz in the indicated compositions, (A) linear scale; (B) logarithmic scale.

FIG. 7: Modules G′ and G″ measured at increasing frequencies in compositions comprising Aloe and pectin and the indicated additives.

FIG. 8: Cellular vitality in Caco2. Optical microscopy image representative of a field showing cells after 3 days in culture (A) in the absence (B) in the presence of a gel composition according to the present invention; (C) measurement of cell viability.

FIG. 9: Modules G′ and G″ measured (A) at increasing frequencies and (B) at 0.1 Hz in compositions comprising Aloe and pectin and the indicated fatty acids.

For the purpose of the present invention, Aloe juice, or Aloe, or Aloe mucilage, means the transparent juice extracted from the pulp or inner part of Aloe leaves, once the rind has been removed and the latex has been washed. In particular, for the purpose of the present invention, the washing has proved to be essential so as to completely remove the exudate, i.e., the latex.

For the purpose of the present invention, pectin means any commercial pectin.

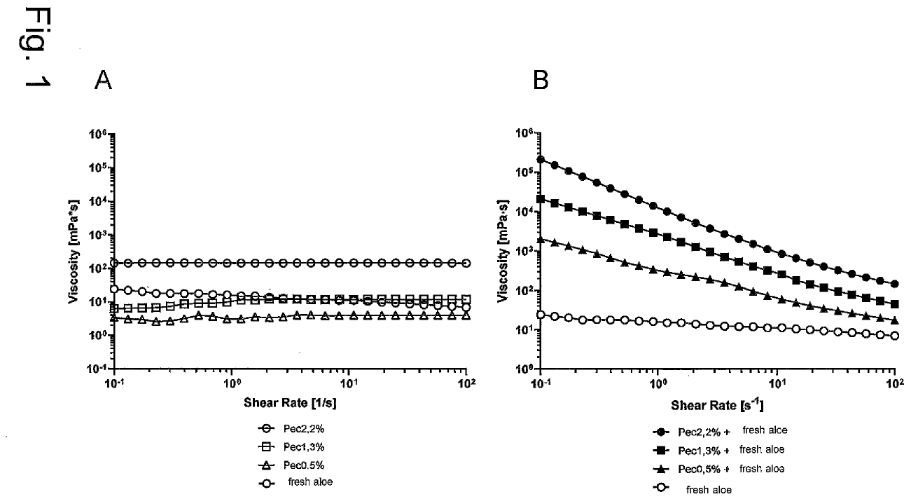

The authors of the present invention have surprisingly demonstrated that a composition comprising Aloe juice and pectin allows obtaining a gel with different and advantageous physical features as compared to what was expected, having known the features of Aloe juice or pectin taken individually. Not only the viscosity of a product comprising Aloe juice and pectin is not the additive viscosity of pectin and Aloe juice, but the combination of the two components modifies the type of material. From the Newtonian fluid, it switches to shear thinning material. In particular, Aloe juice has a power-law behavior (viscosity=K·(shear rate)⁽ⁿ⁻¹⁾), with n=0.8, as obtainable from the graph in FIG. 1A, and is considered as a transition material. Pectin, with an n equal to about 1 for all concentrations tested (FIG. 1A) is called a Newtonian fluid. The product obtained from the combination of Aloe juice and pectin, where pectin is used at the same concentrations at which it was tested not in combination, is strongly shear thinning (n<0.2, FIG. 1B).

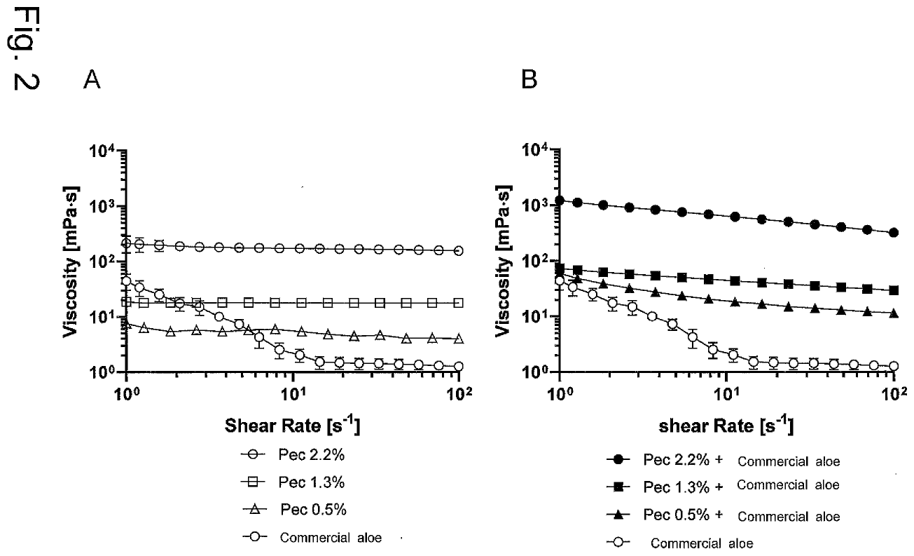

The effect is comparable using fresh Aloe juice, data in FIG. 1, or commercial Aloe juice, data in FIG. 2. After a transition phase, due to the specific features of the juice and the instrumental measurement limit, the viscosity of the commercial Aloe juice settles at a constant and lower value than that of the solutions containing pectin at the indicated concentrations.

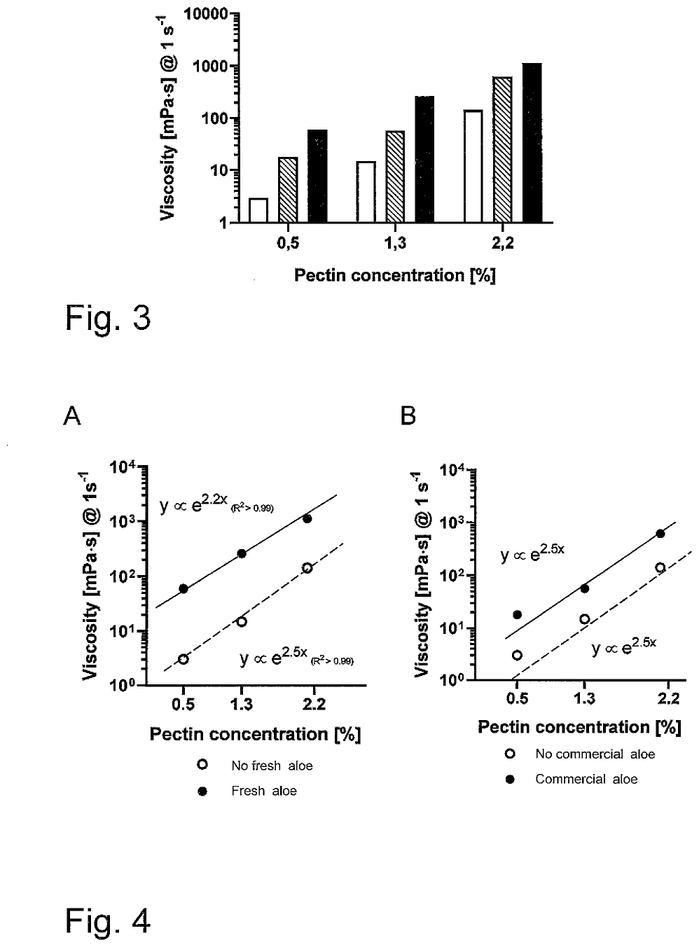

The synergistic effect is clearly visible in the histogram in FIG. 3, where the logarithmic scale viscosity observed in pectin-based products (white columns) at the concentrations 0.5, 1.3 and 2.2% is reported, compared to the viscosity of a product comprising pectin at the same concentrations, with commercial (barred columns) or fresh Aloe juice (black columns) added.

Advantageously, a gel with the rheological features described is obtained by combining Aloe juice and pectin without the need to add crosslinkers and/or reaction initiators. As is known, in order to gel, pH-neutral pectin requires at least one crosslinking initiator and a crosslinker, while Aloe does not gel either in the presence of a reaction initiator or a crosslinker. For the purpose of the present invention, undiluted Aloe vera juice is used, to which pectin and, optionally, further additives and/or active ingredients are added. Said juice is conveniently obtained fresh from the plant, or it is commercial juice. As defined, said juice is deprived of the residual fibrous component and latex.

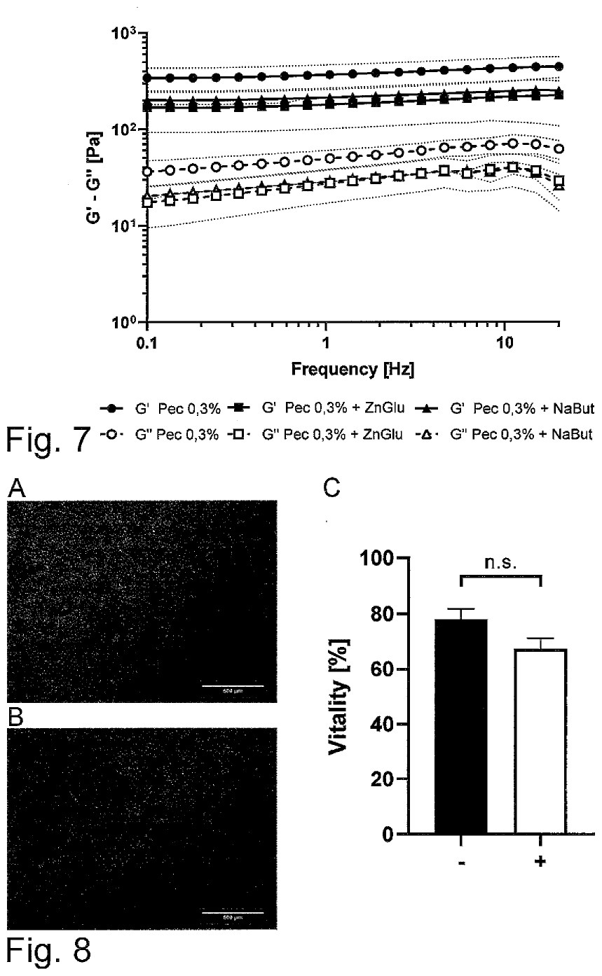

The authors of the present invention have also shown that the composition described herein retains the rheological features of interest even when further active ingredients are added. By way of example, the addition in the composition of Zinc gluconate or Sodium butyrate does not alter the rheological properties thereof, as shown in FIG. 7 and described in example 2 below. Even the addition of vitamins (e.g., 0.006 mg/100 ml) including vitamin B-12, C and D; amino acids (e.g., 0.0025 mg/100 ml) such as L-glutamine; ions (e.g., 0.5 mg/100 ml) such as zinc ion and mixtures of bacteria, including probiotics (e.g., 2.5×10¹⁰ cells/100 ml) such as L. casei, L. plantarum, L. acidophilus, L. delbrueckii, B. longum, B. breve, B. infantis, A. muciniphila and S. boulardii and the like, as well as derivatives thereof; serotonin precursors (25 mg/100 ml) such as 5-http do not alter the measured rheological properties.

In an embodiment, a composition comprising Aloe vera, pectin and at least one short-chain fatty acid and/or salts thereof is claimed. By way of example, said at least one short-chain fatty acid is propionic acid, or is butyric acid.

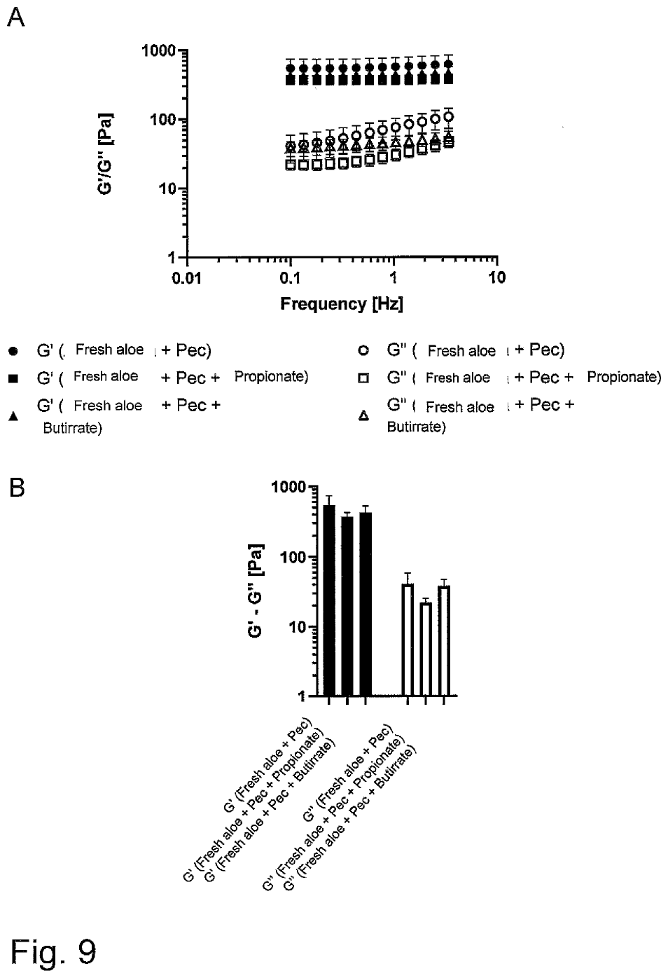

Advantageously, as described in example 4 below and highlighted by the results shown in FIG. 9, said composition maintains the rheological properties measured in the composition Aloe vera+pectin unaltered.

Said composition, having rheological features close to those of the intestinal mucus, is particularly useful for treating inflammatory bowel diseases, where the short-chain fatty acids thus co-formulated better exert the immune-regulatory, microbiota-regulatory, and soothing activity thereof. It is worth noting that the frequency of 0.1 Hz (FIG. 9B) coincides with the frequency of the mechanical stimulations occurring at the intestinal level (Sarna S. K. Gastroenterology 1985; 89:894-913). In a further aspect, a process for obtaining a composition in the form of a gel comprising Aloe juice and pectin is described and claimed, where said process comprises:
providing pectin;
providing Aloe vera juice;
preparing a concentrated solution of said pectin in an aqueous solution, for example 0.9% (w/V) NaCl.
mixing said pectin solution with Aloe vera juice, up to a pectin concentration between 0.1 and 7% (w/V);
after mixing, optionally adding at least one active ingredient selected from the group comprising: vitamins, amino acids, short-chain fatty acids and/or salts thereof, probiotic bacteria;
optionally, adding to said mixture a reaction initiator and/or a crosslinker, such as glucono-delta-lactone and/or calcium carbonate, for example calcium carbonate in a concentration between 0.4 and 1.5% (w/V);
allowing it to rest until the gel forms.

In an embodiment, said process is conveniently implemented using syringes, as described in Podaralla S. et al. 2014, AAPS PharmSciTech, 15: 928-938.

The composition according to the present invention, as such or added with appropriate active ingredients, is advantageously used for treating inflammatory states of the mucosa, for example of the rectal mucosa.

In a preferred form, said composition is applied locally 1 to 5 times a day, preferably twice a day.

In the present invention, dose means the volume of gel which is used in a single application. Said volume is between 10 and 30 ml.

In an embodiment, said active ingredients comprise vitamins, selected from vitamin B-12, vitamin C and vitamin D. In an embodiment, said vitamins are present in a concentration between 0.02 and 1.0 or between 0.03 and 0.20 μg/ml.

In an embodiment, said active ingredients comprise amino acids, preferably L-glutamine, preferably in a concentration between 0.01 μg/ml and 1.0 or between 0.03 and 0.20 μg/ml.

In an embodiment, said active ingredients comprise short-chain fatty acids and/or salts thereof, preferably selected from butyrate, pyruvate and propionate, for example in an amount between 100 and 2000 μg/ml, or between 200 and 1000 μg/ml, or of about 750 μg/ml.

In an embodiment, said active ingredients comprise salts, such as zinc and silver salts, for example in an amount between 0.1 and 150 μg/ml or between 1 and 100 g/ml, or of about 5 μg/ml.

In an embodiment, said active ingredients comprise probiotic bacteria, preferably in the form of tindalized bacteria, for example between 1×10⁸ and 10×10⁸, or 2.5×10⁸ cells/ml. In a preferred form, said composition comprises one or more of L. casei, L. plantarum, L. acidophilus, L. delbrueckii, B. longum, B. breve, B. infantis, A. muciniphila. In an embodiment, said composition comprises VSL #3 (Pharmaceutical Rings) and A. muciniphila.

In an embodiment, serotonin precursors are also present.

In a particularly preferred form, said composition consists of:
Aloe vera juice
pectin, between 0.1 and 3%, or between 0.2 and 1%, or between 0.2 and 0.7%, preferably 0.3% (w/V).

Advantageously, Aloe vera juice is used in the present invention as a natural solvent in which to dissolve the polymer.

The gel described herein is highly customizable, allowing the insertion of one or more additives in the hydrogel matrix in the desired concentration.

The high control over the final mechanical properties makes the gel applicable as an enema with the advantage of releasing the additives locally at the application site.

The following examples have the sole purpose of better illustrating the invention, the scope of which is defined by the claims.

### EXAMPLES

### Example 1: Composition in the Form of a Gel Comprising Aloe and Pectin

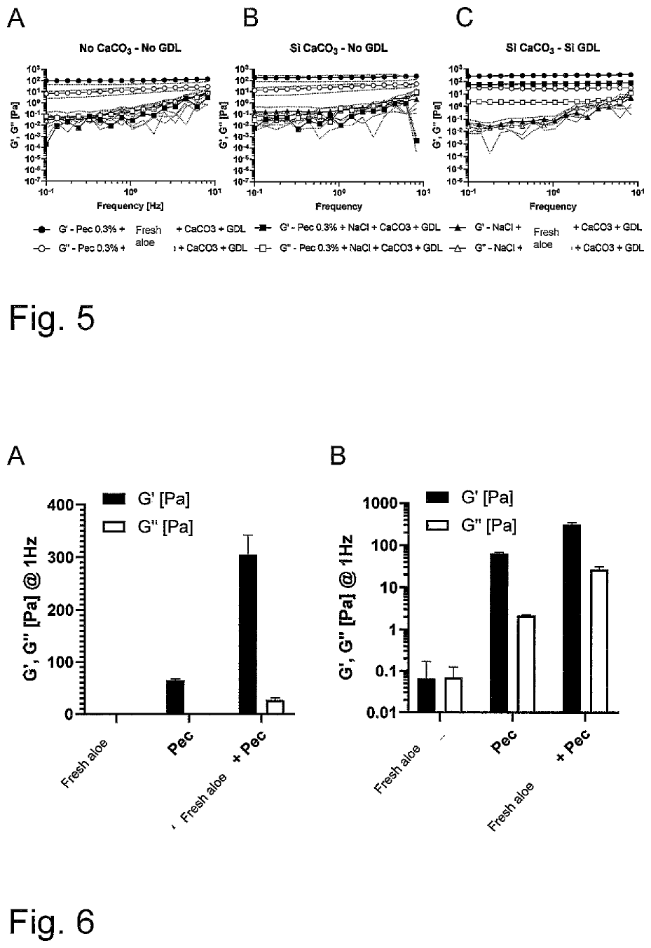

The graphs in FIG. 5 show the conservative module G′ (measures the capacity of the material to store elastic energy when subjected to periodic stresses) and the dissipative module G″ (measures the tendency of the gel to dissipate energy when subjected to periodic stresses) in gels prepared starting from starting solutions of only pectin (squares), only Aloe juice (triangles) and Aloe juice+pectin (circles) in the absence (A) of a crosslinker which is CaCO₃ and of a reaction initiator, which is glucono-delta-lactone (GDL), in the presence (B) of only the crosslinker or in the presence (C) of both the crosslinker and the reaction initiator. A gel is obtained if and only if G′ is greater than G″. The greater G′, the greater the contribution of the elastic component. In the graph in FIG. 5A, obtained without adding either a crosslinker or a reaction initiator, the gel is formed only in the presence of the combination of Aloe juice+pectin (circles). The Aloe juice alone or pectin alone are not capable, in equal concentrations, of giving rise to a gel.

The same is true in the presence of the crosslinker alone (FIG. 5B). Only the presence of crosslinker and reaction initiator leads to the formation of the gel even in the presence of only pectin (FIG. 5C). Aloe juice alone does not create a gel even in the presence of a crosslinker and initiator. The viscoelastic properties of the gel comprising Aloe juice+pectin are greater than the sum of the properties obtained from Aloe juice alone and from pectin alone. In fact, as known from the literature, pectin produces a gel in the presence of crosslinker and GDL with lower viscoelastic properties than those of the gel here obtained by combining Aloe juice+pectin. A gel comprising Aloe juice and pectin has greater viscoelastic properties than those obtained from the individual components (gel without Aloe juice and products without pectin). The viscoelastic properties of the resultant are not simply the sum of those of the gels obtained with the individual components. In addition, Aloe juice alone remains a viscous liquid, throughout the spectrum of frequencies considered.

The same parameters G′ and G″ measured in compositions comprising Aloe, pectin or Aloe+pectin are shown in the histogram in FIG. 6, which clearly highlights the synergistic effect considering only one point of the curve referred to in the graphs in FIG. 5, chosen at 1 Hz, and evaluating the trend of the properties on a linear scale (FIG. 6A) or on a logarithmic scale (FIG. 6B)

The synergistic effect of Aloe juice+pectin is controllable by varying the pectin concentration. The data obtained by measuring the viscosity of samples at increasing concentrations of pectin, in the presence or absence of Aloe juice, show that the relationship describing the changes induced by the pectin concentration is maintained even in the presence of Aloe juice. That is, the synergistic response observed by combining Aloe juice+pectin depends on the pectin concentration according to the relationship valid for the product not containing Aloe juice. The graph in FIG. 4A shows the exponential dependence on the pectin concentration in the absence (dashed line, white circles) or presence (solid line, black circles) of Aloe, fresh juice.

The correlation is also confirmed using commercial Aloe juice, as shown in the graph in FIG. 4B (in the absence of Aloe juice, solid line, white circles, in the presence of commercial Aloe juice, dotted line, black circles).

### Example 2: Addition of Active Ingredients/Additives to the Gel Composition

The graph in FIG. 7 shows how the addition of additives does not modify the demonstrated chemical, physical and mechanical properties for the gel composition comprising Aloe juice and pectin. In particular, gels comprising Aloe juice and 0.3% pectin as described in example 1 were added with ZnGlu or NaBut. The graph shows that the trend of G′ and G″ of the gel with additives does not differ significantly, in the spectrum of the frequencies considered, from the values of G′ and G″ which are obtained from the Aloe juice and pectin gel without additives. This indicates that the presence of additives does not prevent the crosslinking and production of the gel. Therefore, from the application viewpoint it is possible to add additives to the Aloe juice+pectin gel according to the present invention without changing the nature thereof.

### Example 3: Effect of Aloe+Pectin Composition on Cell Viability

Cells of the immortalized Caco2 line of human colorectal adenocarcinoma were plated and exposed to a gel comprising Aloe juice+pectin (0.3% pectin) according to the present invention. As a control, the same cells were maintained in culture without the addition of the gel. FIG. 8 is a representative optical microscopy image of a field obtained after 3 days in culture in the absence (A) or in the presence (B) of the gel. Cell viability was measured by MTT assay, and the results reported in the graph in FIG. 8C. The data show that the gel is not toxic to cells.

### Example 4: Addition of Fatty Acids to the Gel Composition

The graph in FIG. 9A shows how the synergistic effect obtained by combining pectin and fresh Aloe extracted from leaves is maintained adding short-chain fatty acids to the mixture. A composition comprising pectin, fresh Aloe and short-chain fatty acids, such as propionic acid and butyric acid produces a gel as demonstrated by the trend of G′ exceeding G″, with concentrations of propionate (black rectangle curve) and butyrate (black triangle curve) of 2% and 1% respectively. Changing the source of Aloe from fresh to commercial, the results do not change, as shown by the rectangle and white triangle curves.

FIG. 9B shows how the product obtained by mixing fresh Aloe, pectin and short-chain fatty acids (2% propionate or 1% butyrate) mimics the rheological properties of the intestinal mucus (range of about 2-200 Pa), where the measurements are carried out at the frequency 0.1 Hz, which is the frequency of the mechanical stimulations occurring at the intestinal level. By changing the source of Aloe from fresh (black columns) to commercial (white columns), the same effect is observed.

1. A composition in the form of a gel comprising aloe vera juice and pectin in a concentration of 0.1 to 3% (w/V), wherein said aloe vera juice is mucilage deprived of residual fibrous component and latex.

2. A composition according to claim 1, comprising one or more active ingredients selected from the group consisting of:
a vitamin, an amino acid, a short-chain fatty acid, a salt, a probiotic bacterium, and a serotonin precursor.

3. A composition according to claim 1, comprising at least one short-chain fatty acid and/or a salt thereof, in an amount of 100 to 2000 μg/ml.

4. A composition according to claim 2, wherein said probiotic bacteria is selected from the group consisting of L. casei, L. plantarum, L. acidophilus, L. delbrueckii, B. longum, B. breve, B. infantis, A. muciniphila, and A. muciniphila.

5. A composition according to claim 1, further comprising at least one reaction initiator and/or a crosslinker.

6. A method for obtaining a composition in the form of a gel comprising aloe and pectin, wherein said method comprises:
(1) providing pectin;
(2) providing aloe vera juice, wherein said aloe vera juice is mucilage deprived of residual fibrous component and latex;
(3) preparing a concentrated solution of said pectin in an aqueous solution;
(4) mixing said pectin solution with said aloe vera juice, up to a pectin concentration of 0.1 to 3% (w/V), to obtain a mixture;
(5) optionally, adding at least one active ingredient selected from the group consisting of a vitamin, an amino acid, a short-chain fatty acid, and a probiotic bacterium;
(6) optionally, adding a reaction initiator and/or a crosslinker; and
(7) allowing said mixture to rest until a gel forms.

7. A method for the local treatment of an inflammatory state of the mucosa, comprising administering to a subject in need thereof an effective amount of a composition according to claim 1.

8. A method according to claim 7, wherein said mucosa is the rectal mucosa and said composition is administered in the form of an enema.

9. A composition according to claim 1, comprising at least one vitamin, selected from the group consisting of B-12, vitamin C and vitamin D, in a concentration of 0.02 to 1.0 μg/ml.

10. A composition according to claim 1, comprising L-glutamine, in a concentration of 0.01 μg/ml to 1.0 μg/ml.

11. A composition according to claim 1, comprising at least one short-chain fatty acid, selected from the group consisting of butyrate, pyruvate and propionate, in an amount of 100 to 2000 μg/ml.

12. A composition according to claim 1, comprising at least one salt, selected from the groups consisting of a zinc salt and a silver salt, in an amount of 0.1 to 150 μg/ml.

13. A composition according to claim 1, comprising at least one probiotic bacteria, in the form of tindalized bacteria, in an amount of 1×10⁸ to 10×10⁸ cells/ml.

14. A composition according to claim 1, comprising at least one vitamin, selected from the group consisting of B-12, vitamin C and vitamin D, in a concentration of 0.03 to 0.20 μg/ml.

15. A composition according to claim 1, comprising L-glutamine, in a concentration of 0.03 to 0.20 μg/ml.

16. A composition according to claim 1, comprising at least one short-chain fatty acid, selected from the group consisting of butyrate, pyruvate and propionate, in an amount of about 750 μg/ml.

17. A composition according to claim 1, comprising at least one salt, selected from the groups consisting of a zinc salt and a silver salt, in an amount of about 5 μg/ml.

18. A composition according to claim 1, comprising at least one probiotic bacteria, in the form of tindalized bacteria, in an amount of about 2.5×10⁸ cells/ml.

19. A composition according to claim 1, further comprising glucono-delta-lactone.

20. A composition according to claim 1, further CaCO₃.
